(19) United States
(12) Patent Application Publication
(10) Pub. No.: US 2026/0063637 A1
(43) Pub. Date: Mar. 5, 2026

(54) BIOMARKERS FOR DETECTING AND MONITORING BREAST CANCER

(71) Applicant: Sun Jet Biotechnology Inc., Taipei City (TW)

(72) Inventor: Yeou-Guang Tsay, Taipei (TW)

(73) Assignee: Sun Jet Biotechnology Inc., Taipei City (TW)

(21) Appl. No.: 19/299,700

(22) Filed: Aug. 14, 2025

Related U.S. Application Data
(60) Provisional application No. 63/690,538, filed on Sep. 4, 2024.

(51) Int. Cl. G01N 33/574; G01N 33/68

(52) U.S. Cl. CPC G01N 33/57515; G01N 33/57585; G01N 33/6893; G01N 2333/4704; G01N 2800/50; G01N 2800/7028

(57) ABSTRACT

Disclosed is a method for diagnosing a patient's breast cancer (BC) health state, or change in BC health state, or for diagnosing risk of BC or the presence of BC in a patient, comprising detecting, in a plasma sample from said patient, one or more biomarker values that corresponds to thrombospondin-1 (THBS1) or a THBS1-containing complex structure by performing a capillary electrophoresis under non-reducing conditions, and assigning the patient as having or not having BC, or having or not having a change in BC health state, or having or not having a risk of BC based on said biomarker values.

### FIELD OF THE INVENTION

The present invention pertains to a biomarker and a use of biomarker based on thrombospondin 1-containing complex structures for detecting and monitoring breast cancer.

### BACKGROUND OF THE INVENTION

Breast cancer (BC) is the second most common cancer for women, with approximately 2.5% of these cases resulting in death. Despite a high cure rate for localized disease, only 20% of BC cases are diagnosed at an early stage. International studies have reported that the biomarkers CA15-3 and CA27.29 could be used for BC screening, with sensitivities of 30%-57% and 55%-62%, respectively. For the Taiwanese population, the sensitivity of these two markers is quite low, at 5.5% and 6.4% each. Thus, there is still a pressing need of effective biomarkers for BC screening.

Protein complexes, rather than proteins per se, are increasingly being recognized as the primary functional entities in biological activities, even those in bloodstream. This proposition stems from the understanding that proteins rarely act alone, but they often interact with others to form complex structures that mirror specific activities. These complexes can exhibit properties that result solely from the precise assembly of their individual components. For instance, the fibrinogen hexametric complex plays a crucial role in blood clotting, a task that no individual Aa, BB or y subunit could accomplish on its own. There also exists a plethora of mechanisms modifying the structures and subsequently adjusting the functions of circulating protein complexes. Processes specifically associated with diseases like cancers should generate plasma protein complex products that can serve as markers for these diseases.

Biomarker protein complexes, which supposedly make up only a small fraction of the total complex population at early stages, are not easily quantified accurately. For immunochemical methods like ELISA assays, high background noises may mask the signals from these low-abundance complex structures. On the other hand, the use of assays like western blotting is limited by factors such as labor-intensive procedures and inconsistent electro-transfer efficiency for proteins with varying sizes.

### SUMMARY OF THE INVENTION

To address these issues, we have applied automated capillary electrophoresis and immunodetection to develop quantitative assays for biomarker complex structures, and surprisingly found that different species of thrombospondin-1 (THBS1) complex structures can be detected and quantified from human plasma samples, and that their levels and ratios are indicative of the risk of breast cancer.

The present invention provides, in one aspect, a method for diagnosing a patient's breast cancer (BC) health state, or change in BC health state, or for diagnosing risk of BC or the presence of BC in a patient. The method comprises: (i) detecting, in a plasma sample from said patient, one or more biomarker values that corresponds to thrombospondin-1 (THBS1) or a THBS1-containing complex structure by performing a capillary electrophoresis under non-reducing conditions, and (ii) assigning the patient as having or not having BC, or having or not having a change in BC health state, or having or not having a risk of BC based on said biomarker values.

In another aspect, the present invention provides a capture reagent for thrombospondin-1 (THBS1) or a THBS1-containing complex structure, for use in diagnosing a patient's breast cancer (BC) health state, or change in BC health state, or for diagnosing risk of BC or the presence of BC in a patient.

In one further aspect, the present invention provides a kit for performing a method for diagnosing a patient's breast cancer (BC) health state, or change in BC health state, or for diagnosing risk of BC or the presence of BC in a patient, comprising a capture reagent for thrombospondin-1 (THBS1) or a THBS1-containing complex structure, and instructions for performing the method.

In some embodiments, the step of detecting the biomarker values comprises performing an in vitro assay, wherein said in vitro assay comprises a capture reagent for THBS1 or a THBS1-containing complex structure.

In some embodiments, the capture reagent is an antibody.

According to the present invention, a biomarker value may be a signal intensity or normalized signal intensity of any of the peaks P180 and P410, denoted as IP₁₈₀ and IP₄₁₀, respectively, or a ratio of the signal intensities.

In some embodiments, the biomarker values include IP₁₈₀, IP₄₁₀, or a combination thereof.

In some embodiments, the assigning is based on a ratio

$$\frac{{IP}_{410}}{{IP}_{180}}$$

of said biomarker values.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the western blot analyses of plasma samples of BC using polyclonal antibodies against THBS-1 aa 750-850. The asterisk indicates a monomeric 250-kDa THBS-1 species. The white arrow indicates higher MW THBS1 complexes, which correspond to P410 in SimpleWestern analyses. The black arrow shows three components in the medium MW THBS1 complexes, with molecular masses of 300, 380, 430 kDa. The asterisk indicates 250-kDa THBS1 monomer. The bar denotes the range where low MW THBS1 complexes, which supposedly match P180 signals in Simple Western profiles.

FIG. 2 shows the migration profiles of plasma THBS1-containing complexes in capillary electrophoresis for a group of test subjects, including 20 healthy female subjects and 218 breast cancer patients. The numbers to the left represent the signals relative to the height of peak P180. The bottom numbers indicate the migration positions of proteins with corresponding molecular masses. The numbers at the top of each shaded box denote the mass boundaries used for summation of signals in peaks P180 and P410.

FIG. 3 shows the box plots to show statistical analyses of THBS1 multimeric index

$$\frac{{IP}_{410}}{{IP}_{180}}$$

in various subject groups. Seven health/disease conditions were examined, including healthy female subjects, patients with benign diseases, and those with stage 0 to stage IV breast cancer. Each dot on the plot represents the index value for an individual subject.

FIG. 4 shows the receiver operating characteristic (ROC) curves of THBS1 index in distinguishing BC patients from healthy individuals. The vertical axis is sensitivity, and the horizontal axis is (1—specificity) for each THB 1 index cutoff value. The dot on the curve has the maximum of the sum of sensitivity and specificity on the curve, and the number next to the dot denotes the corresponding cutoff value. Its sensitivity and (1—specificity) values are labeled following the cutoff value, respectively.

FIG. 5 shows the summary of the results using the cutoff value 20 for the

$$\frac{{IP}_{410}}{{IP}_{180}}$$

index to access all healthy subjects and breast cancer patients. Black shading indicates values that are twice higher than the cutoff, while the grey shading represents values between 1 and 2 times the cutoff.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereto known to those skilled in the art.

The term “biomarker” used herein refers to a measurable characteristic, either within or external to an organism, that indicates a specific physiological state or the presence of a disease. Biomarkers can be used as indicators for assessing physiological processes, disease progression, drug response, or treatment effectiveness. They may include molecules, cells, tissues, physiological indicators, or imaging features, with their changes often closely associated with disease occurrence, progression, treatment response, etc. Biomarkers have significant applications in clinical diagnosis, prediction, monitoring, and treatment, aiding in improving the accuracy of early disease detection, diagnosis, prognosis assessment, as well as evaluating the effectiveness and safety of treatment regimens.

The term “cancer” used herein refers to a group of diseases characterized by the uncontrolled growth and spread of abnormal cells. These cells can invade and destroy surrounding healthy tissues and can also metastasize to distant parts of the body. Cancer can arise from almost any type of cell in the body and may develop in various organs and tissues. It is typically caused by genetic mutations or other factors that disrupt the normal regulation of cell growth and division.

A biomarker value for the biomarkers described herein can be detected using any of a variety of known analytical methods. In some embodiments, the biomarker value can be detected by performing an in vitro assay, for example, an immunoassay. In one embodiment, a biomarker value is detected using a capture reagent. A biomarker value may also refer to a ratio calculated based on two or more biomarker values, e.g.,

$$\frac{{IP}_{410}}{{IP}_{180}} .$$

As used herein, a “capture agent” or “capture reagent” refers to a molecule that is capable of binding specifically to a biomarker. Capture reagents include but are not limited to aptamers, antibodies, antigens, adnectins, ankyrins, other antibody mimetics and other protein scaffolds, autoantibodies, chimeras, small molecules, an F(ab′)₂ fragment, a single chain antibody fragment, an Fv fragment, a single chain Fv fragment, a nucleic acid, a lectin, a ligand-binding receptor, affibodies, nanobodies, imprinted polymers, avimers, peptidomimetics, a hormone receptor, a cytokine receptor, and synthetic receptors, and modifications and fragments of these.

Thrombospondin-1 (THBS1), encoded by the THBS1 gene in humans, serves as an adhesive glycoprotein that enables cell-to-cell and cell-to-matrix interactions. According to the BioGPS database, this protein is expressed in many different tissues. Thrombospondin-1 from human blood platelets forms a 450-kDa homotrimer under non-reducing conditions and becomes a 150-kDa species upon thiol reduction (Lawler et al., 1978).

The terms “THBS1 multimer,” “THBS1-containing complex structure,” and “THBS1 complex structure” are used interchangeably herein and refer to a protein complex comprising at least one thrombospondin (THBS1) subunit, wherein the at least one THBS1 subunit may be linked to one or more partners (proteins or polypeptides other than THBS1).

In one aspect, the present invention provides a method for diagnosing a patient's breast cancer (BC) health state, or change in BC health state, or for diagnosing risk of BC or the presence of BC in a patient. The method comprises: (i) detecting, in a plasma sample from said patient, one or more biomarker values that corresponds to thrombospondin-1 (THBS1) or a THBS1-containing complex structure by performing a capillary electrophoresis under non-reducing conditions, and (ii) assigning the patient as having or not having BC, or having or not having a change in BC health state, or having or not having a risk of BC based on said biomarker values.

In another aspect, the present invention provides a capture reagent for thrombospondin-1 (THBS1) or a THBS1-containing complex structure, for use in diagnosing a patient's breast cancer (BC) health state, or change in BC health state, or for diagnosing risk of BC or the presence of BC in a patient.

In one further aspect, the present invention provides a kit for performing a method for diagnosing a patient's breast cancer (BC) health state, or change in BC health state, or for diagnosing risk of BC or the presence of BC in a patient, comprising a capture reagent for thrombospondin-1 (THBS1) or a THBS1-containing complex structure, and instructions for performing the method.

In one further aspect, the present invention provides use of a capture reagent for THBS1 or a THBS1-containing complex structure in the preparation of a kit for performing the method as described herein.

According to the present invention, a patient may be assigned as having or not having BC, or having or not having a change in BC health state, or having or not having a risk of BC, based on a higher biomarker value that corresponds to THBS1 or a THBS1-containing complex structure, or a lower biomarker value that corresponds to THBS1 or a THBS1-containing complex structure.

As used herein, a higher (biomarker) value or lower (biomarker) value can refer to a value that is higher or lower compared with a reference level. For example, a lower value can be lower than a reference level by at least 30%, 40%, 50%, 60%, 70%, 80%, 90% or 100%; and higher value can be higher than a reference level by at least 30%, 40%, 50%, 60%, 70%, 80%, 90% or 100%. In some embodiments, a reference level can be a standard (or a threshold) value in a normal individual or a control group. For example, a standard or threshold value can be set based on an average or median level obtained from a cohort of normal subjects. In some embodiments, the cohort of subjects can be a population of normal human subjects (without cancer, or without BC). In addition, a threshold value can be set further based on a desired sensitivity and/or specificity for detecting or diagnosing BC.

According to certain embodiments of the present invention, four species or groups of human plasma THBS1-containing proteins can be resolved using conventional SDS-PAGE and western blotting under non-reducing conditions, including: (i) low MW 140-210K group, (ii) 250K monomeric species, (iii) a medium MW 380-K group having three polypeptides of molecular sizes of about 300, 380, and 430K, respectively, and (iv) the high MW>700K megacomplex.

According to the present invention, human plasma THBS1 complex structures can also be resolved by a capillary gel electrophoresis into two major peaks, including P180 and P410, and corresponds to the low and high MW groups, respectively.

A biomarker value is indicative of a concentration of a biomarker, or a ratio of concentrations of the biomarkers in a sample. A biomarker value of the present invention may be a signal intensity or normalized signal intensity of any of the peaks P180 and P410, denoted as IP₁₈₀ and IP₄₁₀, respectively, or a ratio of the signal intensities. The signal intensity can be measured as the area under the peak. In some embodiments, the signal intensity is measured as the area under the peak in an immunodetection.

The present invention is further illustrated by the following examples, which are provided for the purpose of demonstration rather than limitation.

### EXAMPLES

### 1. Materials and Methods

### 1.1 Reagents for Automated Capillary Electrophoresis

The antibodies used in this study included anti-thrombospondin-1 (Abcam, ab85762), rabbit-derived, and were diluted at a ratio of 1:375. Additionally, antirabbit HRP conjugate (Jackson, 711-035-152) was employed, derived from donkey, and diluted to 1:2,000. The antibody diluent consisted of a mixture of 1% bovine serum albumin (Bionovas, AA0530-0250) in TBST, comprising 20 mM Tris, pH 7.6, 137 mM NaCl, and 0.1% Tween 20. Streptavidin-HRP (GTX27403) was purchased from GeneTex (Hsinchu City, Taiwan) and used at a dilution of 1:1,000. The chemiluminescence reagent was prepared using a 1:1 mix of peroxide solution and luminol reagent from Immobilon Western® Chemiluminescence HRP Substrate (WBKLS0500, Millipore).

### 1.2 Clinical Samples

Blood samples were collected from the participants using BD Vacutainer blood collection tubes with EDTA. The blood samples were processed by centrifugation at 3000×g at 4° C. for 15 minutes in a benchtop centrifuge. Following centrifugation, the supernatant was isolated and saved as the plasma sample, which was then stored at −80° C. until its use in subsequent analyses.

### 1.3 Automated Capillary Electrophoresis and Immunodetection

The reagents and equipment were purchased from ProteinSimple unless stated otherwise. Plasma samples were subjected to a 1:200 dilution, and a 5× Fluorescent Master Mix was added to each sample, with the deliberate omission of any reducing agents. Incubation proceeded at 37° C. for 30 minutes in a water bath. Four microliters of each sample were loaded with electrophoresis buffers for the separation of proteins ranging from 66 to 440 kDa. Both primary and secondary antibody solutions, as well as chemiluminescence reagents and wash buffer, were used per manufacturer's instructions. Using a SimpleWestern system (ProteinSimple, USA), the assays were conducted using the standard protocol as outlined in version 6.1.0 of the accompanying Compass software. The default separation time was set to 25 minutes. The Compass software was used to present the spectra of chemiluminescence signals versus molecular mass. An in-house program coded using Microsoft Visual Basic for Applications (VBA) was developed to generate migration profiles and to quantify signals over specific molecular mass ranges.

### 1.4 Western Blot Analysis

Plasm samples were run on 7% SDS-PAGE gel without reductants. The separated proteins were electrotransferred onto nitrocellulose blotting membrane (Amersham™ Protran™). The blotted membrane was briefly rinsed in Tris-buffered saline (10 mM Tris-HCl PH 7.5, 150 mM NaCl) containing 0.1% (v/v) Tween-20 (TBS-T) before blocking in TBS-T with 1% (w/v) BSA for 1 h at room temperature. Afterward, the membrane was incubated overnight at 4° C. with rabbit anti-thrombospondin-1 (Abcam, ab85762, 1:2000). The membrane was washed three times in TBST for 10 min each before incubation for 1 h with horseradish-peroxidase (HRP)-coupled secondary antibody against rabbit (Jackson, 711-035-152, 1:10000) in TBST with 1% (w/v) BSA. After washing three times in TBST for 1 min each, immunoreactive bands were detected with Immobilon Western® Chemiluminescence HRP Substrate (WBKLS0500, Millipore) and imaging was developed with ImageQuant LAS 4000 (GE Fujifilm). The signals for each indicated region are measured using Image J software.

### 2. Results

2.1 Plasma THBS1 Species in the 410-kDa Peaks in Capillary Electrophoresis Increased Relative to Those in the 180-kDa Peak for Breast Cancer Patients

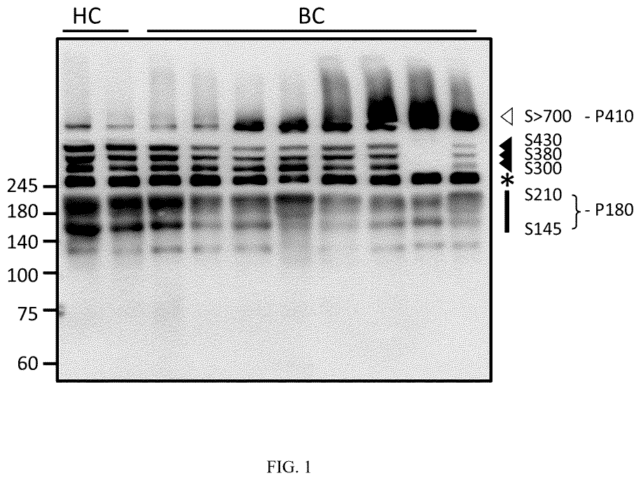

In SDS-PAGE analysis under non-reducing conditions, we identified three distinct groups of species with different molecular weights using specific antibodies against THBS1. These included species ranging from 145 to 210 kDa, species estimated to be around 300, 380, and 430 kDa, and those in the stacking gel exceeding 700 kDa. Particularly in breast cancer patients, the signal intensity for species exceeding 700 kDa was significantly higher compared to that in healthy individuals. Among the eight breast cancer patients analyzed, one patient exhibited similar expression levels for the 145 to 210 kDa species, while the remaining seven patients showed a notable reduction in signals. Additionally, the 300 to 430 kDa species detected in both healthy individuals and breast cancer patients displayed similar expression patterns like the low MW species (FIG. 1). Consequently, we categorized the THBS1 complexes, maintaining their quaternary structure under non-reducing conditions, into three groups: those over 700 kDa high MW, 300 to 430 kDa medium MW, and lastly 145 to 210 kDa low MW ones. The signals of high and low MW groups were estimated using densitometric analyses, and their ratios were determined. For healthy controls (HC), the ratios were below 0.4. For breast cancer patients (BC), all the ratio values were no lower than 0.5 (FIG. 1 and Table 1).

TABLE 1
Densitometric values for high and low MW species measured by Image J, as well
$as their ratios and the \frac{{IP}_{410}}{{IP}_{180}} values for plasma samples$

| | HC | | BC | | | | | | | |
|---|---|---|---|---|---|---|---|---|---|---|
| | 1 | 2 | 1 | 2 | 3 | 4 | 5 | 6 | 7 | 8 |
| S > 700 (A) | 94 | 93 | 116 | 110 | 151 | 163 | 235 | 280 | 271 | 209 |
| S145-S210 (B) | 277 | 240 | 231 | 157 | 205 | 216 | 159 | 157 | 143 | 136 |
| A/B | 0.34 | 0.39 | 0.50 | 0.71 | 0.74 | 0.76 | 1.48 | 1.78 | 1.89 | 1.54 |
| IP410/IP180 | 6 | 7.2 | 11 | 13 | 30 | 26 | 59 | 60 | 71 | 104 |

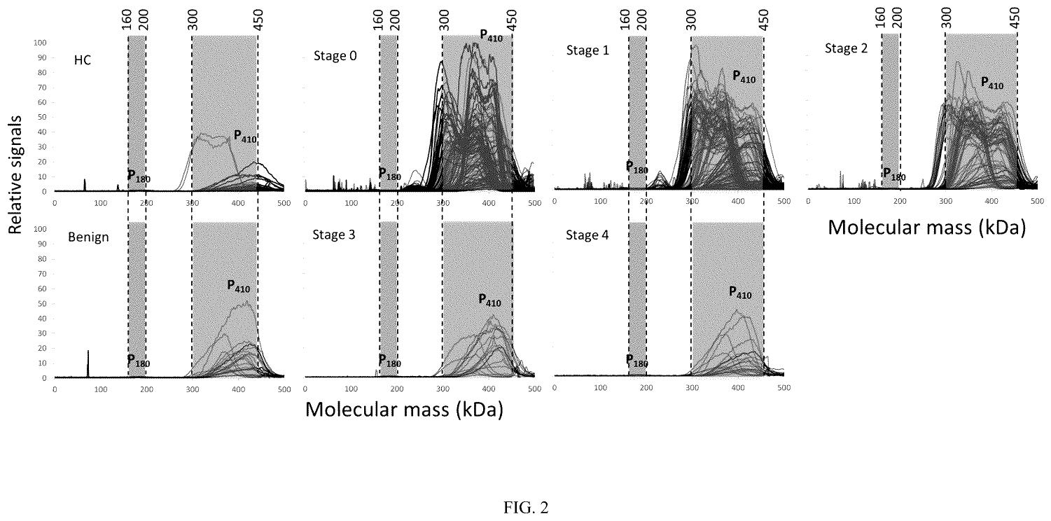

To consistently quantify the changes in circulating THBS1 complex structures in breast cancer (BC) patients, we have opted to utilize capillary electrophoresis and immunodetection, or capillary western blotting (cWB), elegantly implemented in Simple Western system, to analyze plasma samples. For healthy controls, non-reducing cWB analyses revealed two groups of signals. The first group fell within the range of 160 to 200 kDa, with a peak at 180 kDa, and was consequently named P180. The second exhibited signals ranging from 300 to 450 kDa. As most profiles had a peak at 410 kDa, we denoted the signals in this mass range as P410 (FIG. 2). In the case of patients at stage 0 to 2, the P410 signals showed a significant increase compared to the P180 signals. To quantify the changes in these complex species, the

$$\frac{{IP}_{410}}{{IP}_{180}}$$

index has been introduced, where IP₁₈₀ and IP₄₁₀ represent the signal intensities of the respective peaks. This noticeable increase remained consistent despite variations in peak positions of P410 signals among breast cancer patients (FIG. 2). It is notable that, for subjected examined using SDS-PAGE, the ratios with densitometry well reflected the values of

$$\frac{{IP}_{410}}{{IP}_{180}}$$

index (FIG. 1).

We next examined whether the values of this index showed significant differences between healthy controls and breast cancer patients. For healthy subjects, the 25th and 75th percentiles of the

$$\frac{{IP}_{410}}{{IP}_{180}}$$

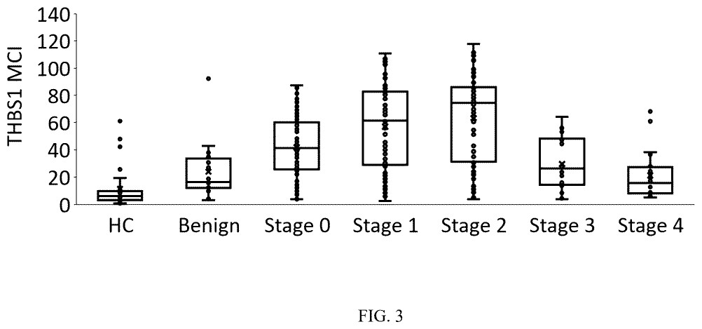

indices were 3 an 10 according to box plot analyses, with a median of 6 and a mean of 12. For patients with benign breast diseases, the median and mean were 18 and 22, respectively. For stage 0˜2 patients, the medians were 44, 62, and 78, and the means were 40, 64, and 74 (FIG. 3). Stage 3 and 4 patients did have significant changes, in comparison with those with benign diseases. Thus, the index apparently rises as the disease progresses from stage 0 to II. Finally, the 25th percentiles of the

$$\frac{{IP}_{410}}{{IP}_{180}}$$

indices for stage 0 to 2 patients were much greater than the 75th percentile of that for healthy controls (FIG. 3), suggesting that the

$$\frac{{IP}_{410}}{{IP}_{180}}$$

index can effectively differentiate breast cancer patients from healthy controls.

### 2.2 the THBS1 Megacomplex Index Demonstrated Good Performance in the Detection of BC Patients

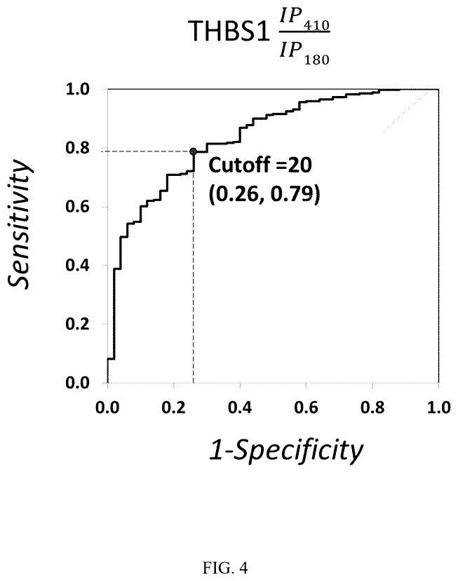

We employed receiver operating characteristic (ROC) curves to assess how well this THBS1 megacomplex index (MCI) worked in breast cancer (BC) patient screening (FIG. 4). Healthy subjects were considered negative, while stage 0 to 2 BC patients were classified as positive. With the significant differences between patients and healthy controls, the ROC curve showed a notable deviation from the diagonal line. This tilting towards the corner signifies that the index acts more like a precise classifier than a random one for distinguishing breast cancer patients. Notably, the data point farthest from the diagonal line showed 79% sensitivity and 74% specificity, with a cutoff value of 20. These results strongly support the potential of the

$$\frac{{IP}_{410}}{{IP}_{180}}$$

index as a reliable biomarker for early breast cancer detection.

### 2.3 the THBS1 Megacomplex Index can Serve as a Valuable Tool in Screening for Early Breast Cancer

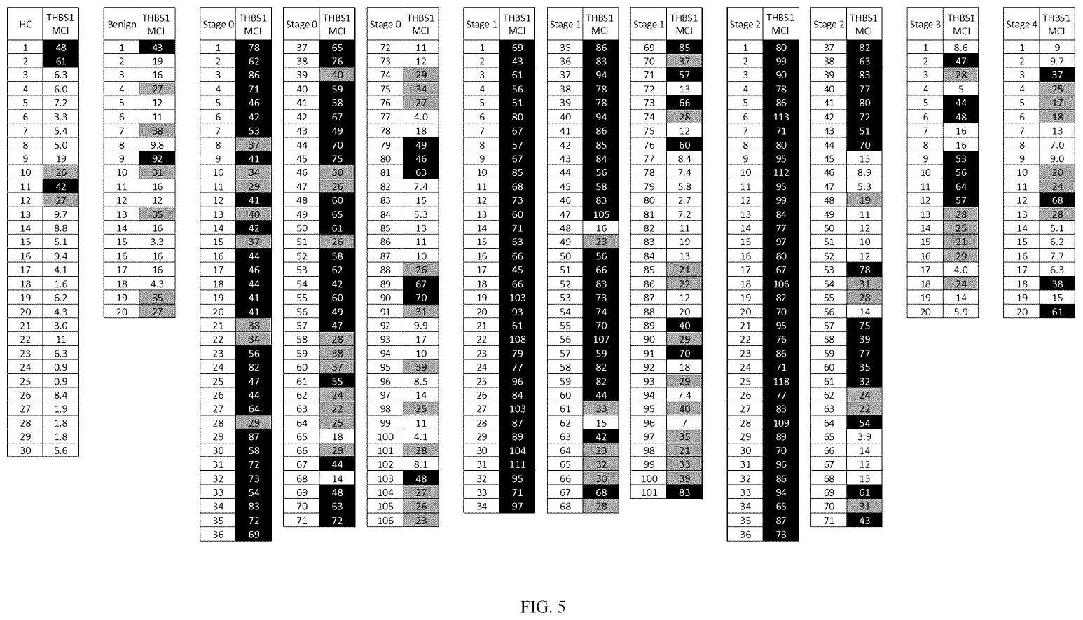

Using the standardized value of 20, we explored how the index THBS1 MCI performed in detecting varying stages of BC within our sample cohort. The analysis revealed that 81%, 83%, and 81% of stage 0, 1 and 2 patients, respectively, could be identified. We also focused on patients with index values exceeding 40, noting that nearly 63% of stage 0 to 2 BC patients had such values (FIG. 5). In conclusion, these outcomes underscore the significance of the

$$\frac{{IP}_{410}}{{IP}_{180}}$$

index as a valuable tool in the screening of early-stage breast cancer.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

1. A method for diagnosing a patient's breast cancer (BC) health state, or change in BC health state, or for diagnosing risk of BC or the presence of BC in a patient, comprising:
detecting, in a plasma sample from said patient, one or more biomarker values that correspond to thrombospondin-1 (THBS1) or a THBS1-containing complex structure by performing a capillary electrophoresis under non-reducing conditions; and
assigning the patient as having or not having BC, or having or not having a change in BC health state, or having or not having a risk of BC based on said biomarker values.

2. The method of claim 1, wherein detecting the biomarker values comprises performing an in vitro assay, wherein said in vitro assay comprises a capture reagent for THBS1 or a THBS1-containing complex structure.

3. The method of claim 2, wherein the capture reagent is an antibody.

4. The method of claim 1, wherein the one or more biomarker values include IP180 and IP410, or combination thereof.

5. The method of claim 1, wherein the assigning is based on a ratio
$$\frac{{IP}_{410}}{{IP}_{180}}$$
of said biomarker values.

6. A kit for performing a method for diagnosing a patient's breast cancer (BC) health state, or change in BC health state, or for diagnosing risk of BC or the presence of BC in a patient, comprising a capture reagent for thrombospondin-1 (THBS1) or a THBS1-containing complex structure, and instructions for performing the method.

7. The kit of claim 6, wherein the method comprising detecting, in a plasma sample from said patient, one or more biomarker values that correspond the THBS1 or the THBS1-containing complex structure by performing a capillary electrophoresis under non-reducing conditions, and assigning the patient as having or not having BC, or having or not having a change in BC health state, or having or not having a risk of BC based on said biomarker values.

8. The kit of claim 6, wherein the capture reagent is an antibody.

9. The kit of claim 7, wherein the one or more biomarker values include IP₁₈₀ and IP₄₁₀, or combination thereof.

10. The kit of claim 7, wherein the assigning is based on a ratio
$$\frac{{IP}_{410}}{{IP}_{180}}$$
of said biomarker values.
